(19) United States
(12) Plant Patent Application Publication
(10) Pub. No.: US 2003/0088896 P1
(43) Pub. Date: May 8, 2003

(54) Peach tree named 'Platanomel'

(76) Inventor: Jose Miguel Arias Lopez, Tudela (ES)

Correspondence Address: CHRISTIE, PARKER & HALE, LLP, 350 WEST COLORADO BOULEVARD, SUITE 500, PASADENA, CA, 91105, US

(21) Appl. No.: 10/185,675

(22) Filed: Jun. 27, 2002

(30) Foreign Application Priority Data
Oct. 23, 2001 (EP) 2001/1657

(51) Int. Cl. A01H 5/00

(52) U.S. Cl. PLT/197

(57) ABSTRACT

A new and distinct variety of Peach Tree having a low chilling requirement, early ripening season producing good quality, yellow fleshed, high firm fruit with attractive color and shape.

### BACKGROUND OF THE INVENTION

The new variety of Peach tree was created in a breeding program by crossing two parents; in particular, by crossing as seed parent a variety designated ‘89-062’ (unpatented) and as pollen parent a variety designated ‘89-067’ (unpatented). Both, female and male, are components of a parent collectIon from a selection made between plants issued from seeds got in a free pollination in a population of different origin done in 1989. Both parental varieties are property and have not been commercialized.

The seeds resulting from this controlled hybridization were germinated in a greenhouse in the spring of 1995 and planted in a field on the farm of La Mogalla in Cartaya (Huelva), Spain, 7°W., 37°N., 45 feet elevation. The seedlings fruited during the spring of 1997, one of which designated as ‘95.04.002-P,’ (unpatented) was selected for its low chilling requirements (250 to 300 hours), early ripening, attractive fruit shape and color. The new variety produces high firm fruit with yellow flesh of good fruit quality. During 1998, the original plant selection was asexually propagated at the above noted location by budding onto a standard Peach rootstock variety designated as ‘GF-677’ (non patented) and a test plot of 4 plants was established.

The new variety has been asexually multiplied several times since 1999 at this location by budding onto the standard Peach rootstock ‘GF-677’ (unpatented) and no incompatibility with the Peach rootstock has occurred following budding. During all asexual reproduction, the characteristics of the original plant have been maintained and no aberrant phenotypes have appeared.

### SUMMARY OF THE INVENTION

The new variety is characterized as to novelty by producing clingstone fruit having an early season ripening date and a low chilling requirement (250 to 300 hours) . Fruit of ‘PLATANOMEL’ are ripe for commercial harvesting and shipment between approximately May 5^{th} to May 15^{th}. These harvesting dates are approximately 12 to 15 days earlier than the harvest dates for the commercial Peach variety ‘MAY CREST’ (unpatented) , and approximately 7 days earlier than the harvest dates for the commercial Peach variety ‘RICH MAY’ (U.S. PP7,432)

### COMPARISON WITH OTHER VARIETIES

‘PLATANOMEL’ differs from Peach trees of ‘MAY CREST’ (unpatented) and ‘RICH MAY’ (U.S. PP7,432) by its fruit size and shape.

‘PLATANOMEL’ produces larger, rounded shaped fruit with a slight apical tip; whereas the fruit of ‘MAY CREST’ are smaller and have a more pronounced apical tip.

The new variety has rounded leaf glands; whereas, the leaf glands of ‘MAY CREST’ are reniform.

The fruit of ‘PLATANOMEL’ are more rounded in shape with a slight apical tip; whereas the fruit shape of ‘RICH MAY’ is ovoid with a strong apical tip.

The fruit surface of ‘PLATANOMEL’ is covered, almost 100%, with a brilliant dark red color; whereas the fruit surface of ‘RICH MAY’ is covered approximately 85% to 90% with a deep red over yellow color.

The new variety produces more homogenous size fruits with a low chilling requirement (250 to 300 hours) ; whereas ‘RICH MAY’ has a chilling requirement of 800 to 900 hours.

### BRIEF DESCRIPTION OF THE DRAWINGS

The accompanying photographic illustrations show typical specimens of the new variety as true to color as is reasonably possible to make in illustrations of this character.

FIG. 1 shows typical flowers of the new variety.

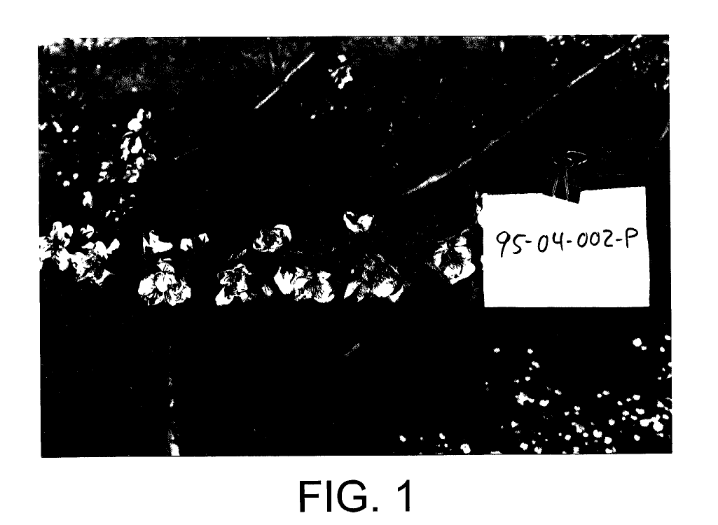

FIG. 2 shows the coloration of dorsal and ventral leaves of the new variety.

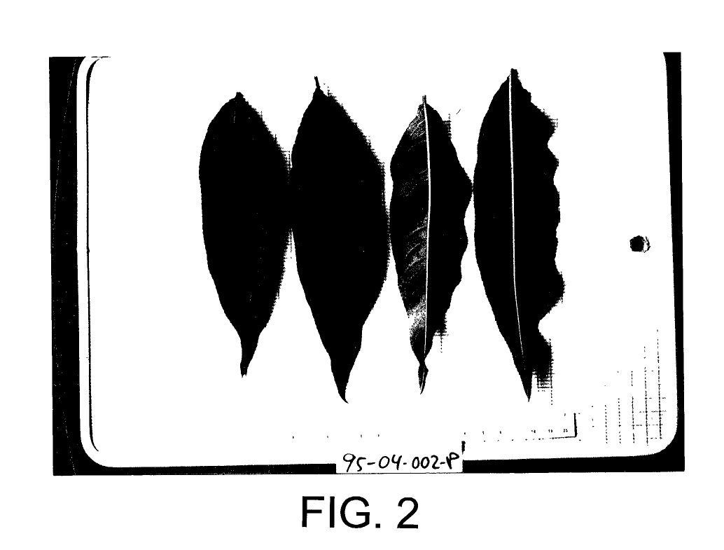

FIG. 3 shows the external coloration of fruit of the new variety when sufficiently matured for harvesting and shipment.

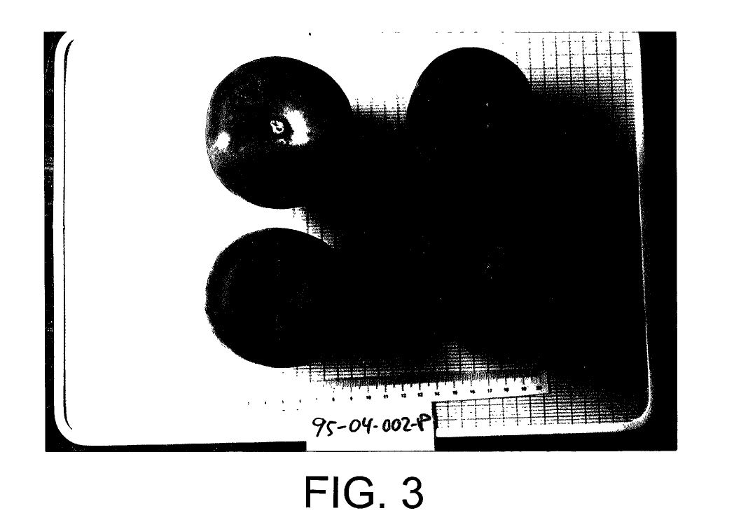

FIG. 4 shows mature fruits of the new variety dissected in the equatorial plane illustrating the characteristics of the flesh and stone.

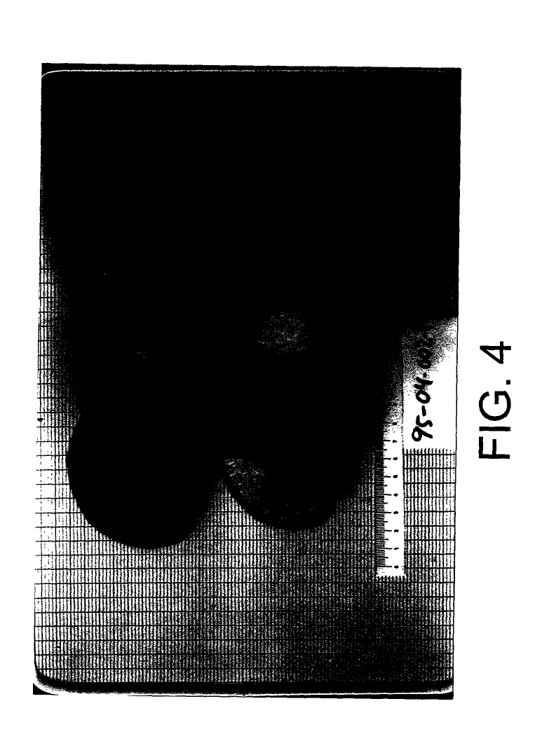

### DESCRIPTION OF THE NEW VARIETY

The following observations and descriptions are of the original seedling tree grown on its own root during the growing years of 1997 to 2001 at the orchard previously described, located near the town of Cartaya, Huelva, Spain.

In this description, it is to be understood that references made to dimensions, sizes, colors, etc. of the botanical and phonological characteristics of the new variety are approximations of averages set forth as accurately as practical.

Phenotypic expression may vary with light intensity and cultural and environmental conditions. The color references used in this description, are to The Royal Horticultural Society Colour Chart (RHSCC) and terminology used in the color descriptions herein refers to plate numbers in said color chart.

### TREE

Size: Average to above average as compared to other common Peach cultivars

Productivity: Approximately 25000 to 28000 Kgrs/Ea.

Form: Upright to upright, spreading in form

Height: Original seedling tree reached approximately 3.4 m at end of 2001 growing season

Width: Original seedling reached approximately 1.90 m at end of 2001 growing season

Current season growth: Approximately 1.0 to 1.2 m

Regularity of bearing: Regular

### TRUNK

Thickness: Approximately 120 mm

Diameter: Approximately 15 cm when measured at a distance above the soil level at end of 2001 growing season

Bark Texture: Moderately rough with numerous scarf skin and flat oval lenticels prosent

Bark Coloration: Grey Group near 201 C to 201 B

### BRANCHES

Size: Considered big for the species

Diameter.—About pb 70.3 mm when measured during the 3^{rd} year after grafting

Surface Texture: Average

Current Season Shoots: Substantially glabrous

Color.—Greyed-Green Group near 194 B to 194 A

Mature Branches: Appearing furrowed on wood which is several years old

Color.—Greyed-green Group near 197 B to 197 A

### LEAF

Size: Average for the species

Length.—Approximately 160 to 200 mm

Width.—Approximately 45 to 60 mm

Thickness.—Approximately 1 to 2 mm

Base Shape: Slightly Oblique

Form: Lanceolate

Tip Form.—Acuminate

Color

Upper Side.—Green Group near 136 B to 139 A

Undersid.—Yellow-Green Group near pb 146 C to 146 B

Textire: Glabrous

Margins: Crenate, generally uniform

Leaf Petiol: Medium in size

Length.—Approximately 1.5 to 2 mm

Width.—Approximately 0.5 to 1.0 mm

### INFLORESCENCE

Flower: Bloom occurs prior to vegetative bud bread; generally double individual flowers at a single node; perfect self-fertile

Blooming Time.—Consisered early in relation to other Peach cultivars

Date of Bloom.—First, February 1^{st}; Full, February 8^{th}

Type:

Flower Diameter.—Approximately 42 to 46 mm at full bloom Length: Approximately 18 to 20 mm Width: Approximately 10 to 20 mm Shape: Round Petal Count: Nearly always 5 Texture: Glabrous Color: Red-Purple Group near 69 D Apex: Petal apices appear domed

Stamens.—Numerous; with polien present; fertile and abundant

FRUIT: Fruit described as would be found in its firm ripe condition at full commercial maturity; first fruit picked on approximately May 5^{th}; last pick of same fruit in growing season 2001 was approximately May 15^{th} in Cartaya, Huelva, Spain conditions

Size: Large; slightly not uniform

Cheek Diameter.—Approximately 70 to 78 mm

Suture Diameter.—Approximately 63 to 72 mm

Form: Rounded with a slight apical tip; generally uniform

Suture: Extending from base to appex, suture appears as a very thin line at same level as skin

Stem cavity size: Considered medium for the species

Width.—Approximately 25 to 35 mm

Length.—Approximately to 40 mm

Depth.—Approximately 15 to 16 mm

Form.—Slightly oval

Fruit Base: Uniform; generally plane and uniform

Fruit Apex: Round with a slight apical tip

Fruit Skin: Average in thickness

Surface Texture.—Medium pubescence

Skin Acidity.—Considered neutral

Tenacious to Flesh.—Yes; at commercial maturity

Tendency to Crack.—Not observed

Skin Coloe.—Covered almost 100% with a brilliant Red Color Group near 46 A to 53 A

Firmness: 4.3 to 4.5 Kg/cm²=Resistance to penetration measured in Kilograms (Kg/cm²); obtained by Penetrometer ROZE Mod. Arbelette, with a 50 mm² section head

Flesh Color: Yellow Group near 12 A to 13 A

Flesh Fibers: Numerous, fine, light red colored fibers present throughout the flesh at maturity

Flesh Texture: Generally melting

Flavor: Sweet; slightly acidic; soluble solids, as °Brix, about 10 to 11

Aroma: Pleasant to medium

Eating Quality: Very good to excellent; well above average when compared to other common commercial varieties

Stone

Attachment.—Clingstone at full commercial maturity

Stone Size.—Considered medium for the species Length: Approximately 27 to 29 mm Width: Approximately 22 to 24 mm Thickness: Approximately 17 to 19 mm

Stone Form.—Ovoid

Stone Color.—Yellow-Orange Group near 20D to 20C

Tendency to Split.—None observed

Kernel.—Ovoid; length approximately 14 to 16 mm; width approximately 10 to 11 mm

USE: Early season maturity producing a highly attractive colored, high firm fruit

KEEPING QUALITY: Fruit stored well up to 20 to 25 days after harvest at temperatures of about 1° C.

RESISTANCE TO INSECTS AND

DISEASE: No particular susceptibilities were noted

SHIPPING QUALITY: Well above average

The new variety of peach tree possesses the characteristics described above as a result of the growing conditions prevailing in Cartaya, Huelva, Spain. It is to be expected that variations in these characteristics may occur when farmed in areas with different climatic conditions, different soil types, and/or varying cultural practices.

I claim:

1. A new and distinct variety of Peach Tree substantially as illustrated and described herein.
